(19) United States
(12) Patent Application Publication
(10) Pub. No.: US 2006/0166860 A1
(43) Pub. Date: Jul. 27, 2006

(54) Zinc transporter compositions for the treatment of cardiovascular diseases

(76) Inventors: Israel Sekler, 84965 Omer (IL); Michal Hersfinkel, 85338 Lehavim (IL); Ze'ev Silverman, 84733 Beer Sheva (IL); Arie Moran, 62261 Tel Aviv (IL)

Correspondence Address: Kevin D McCarthy;Roach Brown McCarthy & Gruber, 1620 Liberty Building, Buffalo, NY, 14202, US

(21) Appl. No.: 10/526,994

(22) PCT Filed: Sep. 8, 2003
(86) PCT No.: PCT/IL03/00742

§ 371 Date: Sep. 12, 2005

(30) Foreign Application Priority Data
Sep. 9, 2002 (IL) 151658

(51) Int. Cl. A61K 38/17; C07K 14/705

(52) U.S. Cl. 514/6; 530/350

(57) ABSTRACT

The present invention relates to the use of ZnT-1, originally described as a zinc transporter, in the regulation of L-type calcium channels (LTCC). In this study, the inventors have unexpectedly demonstrated that ZnT-1 physically interacts with LTCC, regulating its function. Most importantly, the inventors have shown that ZnT-1 can regulate intracellular Ca₂₊ influx, and thus, its intracellular concentration. This is the first demonstration of a natural blocker for LTCC, and it is a promising breakthrough as a potential agent to be used in the treatment and/or prevention of cardiovascular diseases and related indications.

### FIELD OF THE INVENTION

The present invention relates to the use of a polypeptide, involved in zinc transport, in the regulation of L-type calcium channels for the treatment and/or prevention of cardiovascular diseases.

### BACKGROUND OF THE INVENTION

All publications mentioned throughout this application are fully incorporated herein by reference, including all references cited therein.

According to the WHO, cardiovascular disease (CVD) was responsible for one third of global deaths in 1999. In developed countries, CVD is regarded as the major cause for morbidity and mortality. By 2010, CVD is estimated to be the leading cause of death in developing countries as well. Therefore, the search for more effective drugs to prevent and treat CVD is highly desirable.

Calcium channels play a key role in the physiology and pathophysiology of the cardiovascular system. Consequently, they are crucial pharmacological targets in the treatment of numerous cardiovascular diseases. For example, drugs aimed at reducing hypertension act by blocking the calcium influx pathway. However, a major drawback using these medicines is their relative high toxicity and lack of specificity.

The L-type calcium channels (LTCC), or sarcolemmal L (long lasting) channels, are typical calcium channels found in the myocardium and involved in calcium-induced calcium release. The LTCC is responsible for responding to the well-known Ca⁺² channel blockers dihydropyridines. In fact, these channels are also known as dihydropyridine receptors, because of their high affinity to this class of Ca⁺² channel blockers.

Zinc (Zn²⁺) is among the most abundant trace elements in mammals. It is an essential micronutrient that has numerous structural and regulatory functions, including its interaction with zinc finger domains and acting as a cofactor in many enzymes [Vallee B. L. et al., Physiol. Rev. 73:79-118 1993]. Zinc ions also bind and specifically modulate the activity of many membrane receptors, transporters and channels [Choi D. W. et al. (1998) Ann. Rev. Neurosci. 21:347-75; Huang E. P. (1997) Proc. Natl. Acad. Sci. USA 94:13386-7]. The LTCC is one of the major routes of zinc entry in the brain [Sensi, S. L. et al. (1997) J.Neurosci. 17:9554-64]. In other cell types, such as glia or cardiomyocytes, the role of LTCC is even more prominent, being the only identifiable zinc influx pathway in these cells [Atar, D. et al. (1995) J. Biol. Chem. 270:2473-7]. The distribution of free zinc across the plasma membrane resembles that of calcium, since it has a large gradient, exceeding 5 to 6 orders of magnitude [Choi, D. W. and J. Y. Koh. (1998) Annu. Rev. Neurosci. 21:347-75]. In fact, opening of the calcium channels leads to a massive zinc influx [Atar (1995) id ibid.].

In the brain, the extracellular concentration of zinc is extremely high. As a result, massive release of synaptic zinc occurs during ischemia, which permeates into neurons, and most likely is the major factor in ischemic neuronal cell death [Choi D. W. et al. (1998) id ibid.; Choi D. W. et al. (1988) Neuroscience 24:67-79; Kim Y. H. et al. (1999) Neuroscience 89:175-82; Koh, J. Y. et al. (1996) Science 272: 1013-6]. The dynamic nature of zinc effects is further demonstrated in diverse pathophysiological syndromes including myocardial infarction, hepatic renal failure, and neoplasia. In such cases, striking changes occur in zinc plasma concentration and the rate of disappearance of these changes is considered a reliable prognostic marker [Vikbladh I. (1950) Scand. J. Clin. Lab. Invest. 2:143-148].

Sudden and de-regulated increase in intracellular zinc is highly cytotoxic. Therefore, maintenance of low intracellular zinc concentration is a very important function. This maintenance is achieved either through systems that mediate extrusion of zinc against its high electrochemical gradient, or through systems that down-regulate zinc influx via the LTCC. Recent studies have linked a newly discovered family of putative zinc transporters, ZnT-1 through 4, to the maintenance of Zn²⁺ homeostasis and protection against zinc toxicity by a yet unidentified mechanism. In fact, the first putative mammalian zinc transporter (ZnT-1) was cloned through functional complementation of a mutant BHK cell line sensitive to extracellular zinc [Palmiter, R. D. and S. D. Findley (1995) EMBO J. 14:639-49]. ZnT-1 has a putative structure of six membrane spanning domains and it shares homology with a yeast zinc resistance gene and with a cobalt transporter [Palmiter (1995) id ibid]. So far, the functional mechanism of ZnT-1 remains unknown.

In the examples hereby described, ZnT-1 down-regulates zinc influx through the LTCC, supporting a regulatory role rather than a direct transport activity.

It is an object of the invention to provide the use of ZnT-1 as a protein responsible for upholding Ca homeostasis, through its interaction with the LTCC. In addition, the inventors have unexpectedly shown that ZnT-1, originally described as a Zn²⁺ transporter, can function as a blocker of the LTCC and thereby regulate intracellular Zn²⁺ concentration.

It is another object of the present invention to provide a pharmaceutical composition for the treatment of diseases that affect LTCC function, typically cardiovascular diseases, comprising ZnT-1 as the active agent. These and other objects of the invention will become apparent as the description proceeds.

### SUMMARY OF THE INVENTION

In a first aspect, the present invention relates to ZnT-1 and/or fragments thereof for use as a cellular LTCC blocker, which blocker has the properties of a modulator of LTCC function, and of a modulator of intracellular calcium concentration.

The present invention provides ZnT-1 and/or fragments thereof for use as an LTCC blocker, in the treatment and/or prevention of cardiovascular diseases and related indications, particularly hypertension and stroke.

In a first embodiment, said LTCC blocker, or ZnT-1 and/or fragments thereof, is active in vascular cells, cardiomiocytes, neurons and glia.

In a second aspect, the invention relates to a composition for blocking L-type calcium channels, comprising as active agent ZnT-1 and/or fragments thereof. As mentioned above, ZnT-1 can regulate LTCC function, as well as modulate intracellular calcium concentrations.

In a first embodiment, the composition is for use in the treatment and/or prevention of cardiovascular diseases and related indications. Preferably the composition of the invention is for use in the treatment and/or prevention of hypertension and stroke.

In yet another aspect, the invention relates to a method for treating and/or preventing cardiovascular disorders in a patient in need of such treatment.

In a first embodiment, the method of the invention comprises administering to the patient a LTCC blocker or ZnT-1 and/or fragments thereof, or a composition comprising the same, wherein said blocker is capable of modulating the intracellular concentration of calcium and of regulating LTCC function.

ZnT-1 and/or fragments thereof may also be used in the preparation of a pharmaceutical composition for the treatment and/or prevention of cardiovascular diseases and related indications, preferably hypertension and stroke.

### BRIEF DESCRIPTION OF THE FIGURES

FIG. 1: Fura-2 fluorescence is dependent on Zn²⁺ concentration

The signal in this graph reflects changes in fluorescence signal measured at 340/380 nm of excitation and 510 nm emission, which is proportional to intracellular zinc levels, throughout a period of 600 seconds.

FIG. 2: Zn²⁺ influx is inhibited by a L-type Ca channel blocker

HEK 293 cells were exposed to 400 μM zinc with or without 1 μM nimodipine. The LTCC blocker inhibited the influx of zinc into the cells. The signal in this graph reflects changes in fluorescence signal measured at 340/380 nm of excitation and 510 nm emission, which is proportional to intracellular zinc levels, throughout a period of 300 seconds.

FIG. 3: ZnT-1 does not affect zinc efflux

Graph showing the monitoring of Zinc efflux in cells transfected with ZnT-1 or not. The results show that in cells transfected with ZnT-1, zinc efflux was similar to control, non-transfected cells.

Inset: cells were exposed to 400 μM Zn, and fluorescent signal was enhanced. The signal was eliminated by TPEN an intracellular zinc (and not calcium) chelator. Indicating that fluorescent signal results indeed due to changes in [Zn]ᵢ.

FIG. 4a-c: Expression of ZnT-1 Reduces Zinc Accumulation

FIG. 4a: Control and ZnT-1 transfected HEK 293 cells were exposed to 400 μM zinc, and the rate of zinc influx was monitored over time.

FIG. 4b: Expression of ZnT-1 was evaluated by Western Blot. The two bands reflect the presence of glycosylated and unglycosylated forms of ZnT-1. Endo-F (EF) was used to cleave the glycosyl residues of ZnT-1. Such treatment resulted in merging of the two bands into one, indicating that the presence of the two bands is indeed related to different glycosylation states of the protein.

FIG. 4c: Histogram representing the rates of zinc influx into HEK293 cells transfected with LTCC alone or in combination with ZnT-1. A 5 fold reduction in the rate of zinc permeation through LTCC can be observed in cells co-transfected with both LTCC and ZnT-1 in comparison to cells transfected with LTCC alone.

FIG. 5a-b: ZnT-1 expression inhibits Zn²⁺ influx into cells

FIG. 5a: Control and ZnT-1 transfected PC 12 cells, expressing endogenous L-type Ca channels, were superfused with Ringer solution containing 400 μM of marker cation Zn²⁺. The cells were then depolarized by high K⁺ in the presence of zinc.

FIG. 5b: Rate of zinc influx into the cells. After depolarization, the rate of zinc influx was three-fold slower in cells expressing ZnT-1 vs. control cells.

FIG. 6: Co-expression of ZnT-1 and L-type Ca channels in HEK 293 cells reduces zinc influx

The graph shows zinc influx (measured by fluorescence) in cells transfected with L-type Ca channels (LTCC) alone (squares), cells transfected with LTCC in combination with ZnT-1 (diamonds), and control non-transfected cells (circles). Transfection with LTCC increased the zinc influx by about three-fold. Co-expression of LTCC and ZnT-1 caused reduction in the cation influx to levels comparable to control.

FIG. 7: LTCC expression is not changed by ZnT-1 expression

Immunoblot showing that LTCC expression is not changed by ZnT-1 expression in HEK 293 cells transfected with LTCC, and PC12 cells endogenously expressing LTCC, in the presence or absence of ZnT-1.

FIG. 8a-b: ZnT-1 expression inhibits Ca²⁺ influx into cells

FIG. 8a: Control and ZnT-1 transfected PC 12 cells, expressing endogenous L-type Ca channels, were superfused with Ca-free Ringer solution. After addition of 1.8 mM calcium, the cells were depolarized by high K⁺ for opening of the LTCC (arrows) and the levels of Ca start to decrease compared to control.

FIG. 8b: Histogram of the rate of Ca influx into control and ZnT-1 transfected cells.

FIG. 9a-b: ZnT-1 expression is induced by Zn in cultured brain astrocytes

FIG. 9a: Astrocytes were treated with sub-lethal amounts (20 and 50 μM) of Zn²⁺ for 12 hours. Histogram shows the level of ZnT-1 expression in cells treated with 20 μM and 50 μM of Zn²⁺ or with EDTA. ZnT-1 expression is induced by Zn²⁺ in a dose-dependent manner.

FIG. 9b: Western Blot of ZnT-1 expression.

FIG. 10a-b: Zinc influx in cells induced to express ZnT-1 protein is reduced in a concentration-dependent manner

FIG. 10a: Astrocytes were pre-treated with zinc at the indicated concentrations, and subsequent rates of zinc influx were determined. The histogram represents the relative rate of zinc influx. Reduced zinc influx correlates with higher expression of ZnT-1.

FIG. 10b: Graph demonstrating the change in intracellular zinc following zinc application, in pre-treated and control cells.

FIG. 11: ZnT-1 and LTCC physically interact

Western blot showing that immunoprecipitation with ZnT-1 antibody followed by immunoblot with LTCC antibody reveals a band, in both cardiac tissue and in HEK cells co-transfected with LTCC and ZnT-1, which represents the complex between the two proteins. As shown, co-expression of the two proteins in HEK 29 cells resulted in much higher protein levels (see HEK cells).

### DETAILED DESCRIPTION OF THE INVENTION

For the purposes of clarity, the following terms are defined herein:

LTCC: L-type calcium channel, sarcolemmal L (long lasting) channel.

ZnT-1: Zinc transporter 1, LTCC blocker.

Cardiovascular disease (CVD): The term cardiovascular disease includes hypertension (high blood pressure), coronary heart disease (heart attack), cerebrovascular disease (stroke), peripheral vascular disease, heart failure, rheumatic heart disease, congenital heart disease and cardiomyopathies.

The inventors have surprisingly discovered that ZnT-1 directly interacts with the LTCC, and regulates calcium influx into the cell. Thus, ZnT-1 plays a key and novel physiological role in calcium homeostasis. As shown in the following examples, ZnT-1 is the first natural blocker of the LTCC. The clinical implications of these findings are significant since calcium influx through these channels is implicated in the pathogenesis of cardiovascular abnormalities such as high blood pressure at the vascular smooth muscle level, contractile and electrophysiology abnormalities in the myocardial myocytes and specialized conduction cells at the cardiac cell level.

Thus, in a first aspect, the present invention relates to ZnT-1 and/or functional fragments thereof for use as a cellular LTCC blocker, which blocker has the properties of a modulator of LTCC function, as well as of a modulator of intracellular calcium concentrations.

A LTCC modulator may enhance or inhibit the LTCC function. A modulator of intracellular calcium concentrations may regulate the influx or outflux of this ion from the cell, in order to maintain the appropriate concentrations. Thus, a LTCC modulator, or ZnT-1, may trigger an increase or a decrease in the intracellular concentration of calcium.

Thus, the present invention provides ZnT-1, and/or functional fragments thereof, for use as an LTCC blocker, in the treatment and/or prevention of cardiovascular diseases and related indications, particularly hypertension and stroke.

ZnT-1 refers to the protein encoded by the gene ZnT-1, originally described as a zinc transporter [Palmiter and Findley (1995) id ibid.; GenBank Accession No. U17132].

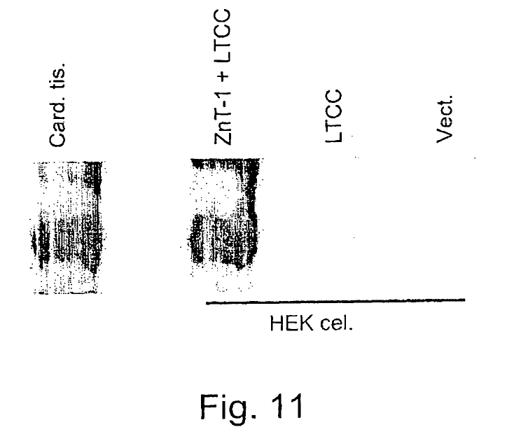

By the term functional fragments it is meant any fragment that has the properties of a modulator of LTCC function, as well as of a modulator of intracellular calcium concentrations. In FIG. 11, the inventors demonstrate that ZnT-1 and LTCC physically interact. Thus, the ZnT-1 fragment responsible for this interaction is also considered herein a functional fragment.

The term ZnT-1 should be considered herein as also comprising functional fragments thereof.

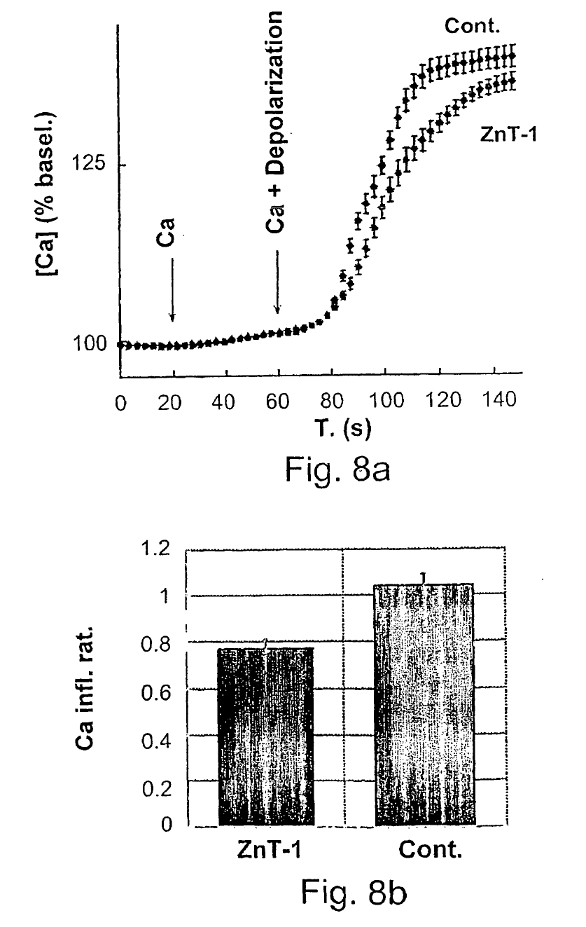

As shown in the following Examples, cells expressing ZnT-1 can block Ca²⁺ (FIG. 8) influx into the cells in a similar fashion to nimodipine, a dihydropyridine that acts as a LTCC blocker.

The inventors have shown that ZnT-1 can lower intracellular zinc and furthermore, that ZnT-1 acts by downregulating ion influx through the LTCC. Unexpectedly, the inventors have shown, in FIG. 11, that ZnT-1 and LTCC physically interact. This is an original finding, never suggested in the existing prior art. These findings led the inventors to propose that ZnT-1 plays a significant role in the regulation of the LTCC, and in the mechanism for the lowering of intracellular calcium.

Furthermore, the main known role of the LTCC is the regulation of calcium permeation and thereby the regulation of key homeostatic functions such as blood pressure. The results presented herein thus indicate, for the first time, that ZnT-1 is responsible for the regulation of intracellular calcium concentrations and thus, it is likely involved in regulating such processes.

There are many examples for ion permeation pathways that have different selectivity for different ions. The results shown in FIG. 8 unambigiously demonstrate the role of ZnT-1 in the regulation of calcium permeation through this very important pathway. Moreover, under conditions of high extracellular concentration of Zn²⁺, the expression of ZnT-1 is induced. Thus, ZnT-1 seems to be the physiological regulator and blocker of LTCC function.

In a first embodiment, said LTCC blocker, or ZnT-1 and/or functional fragments thereof, is active in vascular cells, cardiomiocytes, neurons and glia. This function is particularly relevant in astrocytes, embryonic kidney cells and pherochromocytoma cells.

As demonstrated in Example 7, said LTCC blocker, or ZnT-1 and/or functional fragments thereof, may be induced intracellularly upon exposing the cells to concentrations of from about 1 μM to 1 mM of zinc. Preferably, an induction is observed when the concentration of zinc is about 200 μM.

In a second aspect, the invention relates to a pharmaceutical composition for blocking L-type calcium channels, comprising as active agent ZnT-1 and/or functional fragments thereof. As mentioned above, ZnT-1 can regulate LTCC function, as well as modulate intracellular calcium concentrations.

This is of particular importance in cases of hypertension or stroke, where LTCC plays a critical pathophysiological role.

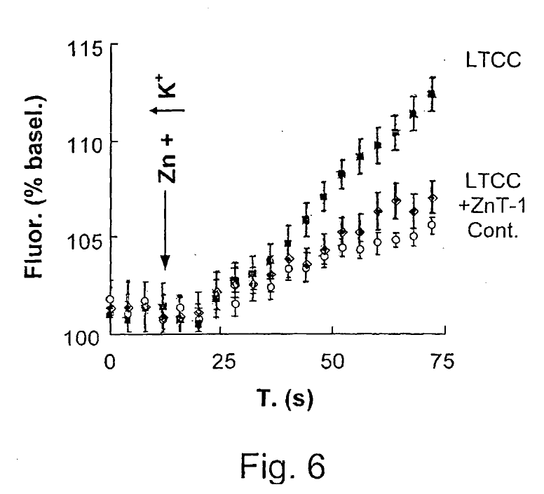

As shown in Example 5, ZnT-1 is also a modulator of intracellular zinc concentration. Cells expressing LTCC in the presence of ZnT-1 have a rate of zinc influx about three fold slower than in its absence (FIG. 6).

In a first embodiment, the composition is for use in the treatment and/or prevention of cardiovascular diseases and related indications. Preferably the composition of the invention is for use in the treatment and/or prevention of hypertension and stroke.

Likely additives to the composition comprise pharmaceutically acceptable adjuvants, carriers, diluents or excipients. In addition, the composition may comprise additional pharmaceutically active components.

Acceptable carriers, excipients, or stabilizers are non-toxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol and sorbitol; slat-forming counter-ions such a sodium; and/or non-ionic surfactants such as Tween, Pluronics™ or polyethylene glycol (PEG).

The pharmaceutical compositions of the invention can be prepared in dosage unit forms. The dosage forms may also include sustained release devices. The compositions may be prepared by any of the methods well known in the art of pharmacy. Such dosage forms encompass physiologically acceptable carriers that are inherently non-toxic and non-therapeutic. Examples of such carriers include ion exchangers, alumina, aluminum stearate, lecitin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of the composition include polysaccharides such as sodium carboxymethylcellulose or methylcellulose, polyvinylpyrrolidone, polyacrylasts, polyoxyethylene-block polymers and PEG. For all administrations, conventional delivery forms are suitably used. Such forms include for example, microcapsules, nano-capsules, liposomes, plasters, inhalation forms, nose sprays, sublingual tablets, and sustained-release preparations.

The preparation of pharmaceutical compositions is well known in the art and has been described in many articles and textbooks, see e.g., Remington's Pharmaceutical Sciences, Gennaro A. R. ed., Mack Publishing Company, Easton, Pa., 1990, and especially pages 1521-1712 therein, fully incorporated herein by reference.

The magnitude of a therapeutic dose of the composition will of course vary with the group of patients (age, sex, etc.), with the stage of the disease, and with the route of administration. In any case, the attending physician will determine the therapeutic dose.

Any suitable route of administration may be employed for providing a mammal, especially a human, with an effective dosage of an LTCC blocker or ZnT-1, or the pharmaceutical composition of the invention. Suitable routes of administration include oral, topical application or invasive administration techniques. The term “invasive” as used herein is to be taken to mean intra-cerebral, intraspinal, intramuscular, intravenous, intraperitoneal, subcutaneous, intra-articular, intrasynovial or intrathecal administration.

An “effective amount” of ZnT-1 or the composition of the invention to be employed therapeutically will depend, for example, upon the therapeutic objectives, the route of administration, and the condition of the patient. Accordingly, it will be necessary for the therapist to titer the dosage and modify the route of administration as required, to obtain the optimal therapeutic effect. Typically, the clinician will administer ZnT-1 until a dosage is reached that achieves the desired effect.

In Example 8, the inventors show that astrocytes induced to express ZnT-1 have much reduced rates of zinc influx.

In yet another aspect, the invention relates to a method for treating and/or preventing cardiovascular disorders in a patient in need of such treatment.

In a first embodiment, the method of the invention comprises administering to the patient a LTCC blocker or ZnT-1 and/or functional fragments thereof, or a composition comprising the same, wherein said blocker is capable of modulating the intracellular concentration of zinc and/or calcium and of regulating LTCC function.

Preferably, said treatment is for a patient suffering from cardiovascular disease, in particular hypertension.

“Treatment” refers to therapeutic treatment. Those in need of treatment include those already with the disease or disorder, whether at clinical or pre-clinical stage.

ZnT-1 and/or functional fragments thereof may also be used in the preparation of a pharmaceutical composition for the treatment and/or prevention of cardiovascular diseases and related indications, preferably hypertension and stroke.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Zinc influx, driven by steep inward transmembrane zinc gradients, plays a fundamental role in zinc signalling and in pathophysiologies linked to cellular accumulation of toxic zinc in cells. ZnT-1, an ubiquitously expressed protein, was shown to confer resistance against toxic zinc. However, the mechanism linking ZnT-1 to lowering of [Zn]ᵢ is not understood. Here the inventors addressed this issue using fluorescent measurements of zinc transport in cells heterologously expressing ZnT-1. Rates of zinc efflux in HEK 293 cells expressing ZnT-1 were not accelerated indicating that ZnT-1 may be involved in regulating influx rather than zinc extrusion. Expression of ZnT-1 in PC12 cells resulted in a 3-fold reduction in zinc influx mediated through LTCC, a major route for zinc influx, without changing LTCC expression. Heterologous co-expression of ZnT-1 and LTCC in HEK 293 cells also resulted in a 3 fold reduction in the rate of zinc influx, further indicating that ZnT-1 modulates zinc permeation through LTCC. Finally, ZnT-1 regulation of the LTCC is shown to regulate calcium permeation via this channel, using ZnT-1 transfected PC 12 cells.

These findings therefore indicate that ZnT-1 modulates cation permeation through LTCC and, therefore, plays a general role in regulating not only zinc, which was used in the following examples mainly as a cation marker, but most importantly, in calcium permeation. The ubiquitous nature of ZnT-1 and LTCC distribution, taken together with the key role of the latter in cellular ion homeostasis, underscores the importance of such regulation.

Experimental Procedures

General Methods in Molecular Biology

A number of methods of the molecular biology art are not detailed herein, as they are well known to the person of skill in the art. Such methods include PCR cloning, expression of cDNAs, analysis of recombinant proteins or peptides, transformation of bacterial and yeast cells, transfection of mammalian cells, and the like. Textbooks describing such methods are, e.g., Sambrook et al., Molecular Cloning, A Laboratory Manual, Cold Spring Harbor Laboratory, ISBN: 0879693096, 1989; Current Protocols in Molecular Biology, by F. M. Ausubel, ISBN: 047150338X, John Wiley & Sons, Inc. 1988. These publications are incorporated herein in their entirety by reference. Furthermore, a number of immunological techniques are not in each instance described herein in detail, as they are well known to the person of skill in the art. See, e.g., Current Protocols in Immunology, Coligan et al. (eds), John Wiley & Sons. Inc., New York, N.Y.

Cell Culture and Transient Transfections

PC12 and human embryonic kidney (HEK) 293 cells were grown in DMEM medium (Sigma) as described [Montrose Rafizadeh, C. et al. (1997) J. Cell Physiol. 170:299-308], and seeded on cover slides for 1-2 days prior to measurements.

HEK 293 cells were transiently transfected with 3 μg of plasmid containing ZnT-1 (kindly provided by Dr. R. D. Palmiter, Howard Hughes Medical Institute, University of Washington, Seattle, Wash.) or vector alone, using the calcium precipitation method as described [Sekler, I. et al. (1995) J Biol. Chem. 270:11251-6]. Pherochromocytoma PC12 cells were co-transfected with ZnT-1 or EYFP (Clontech) for control, using the Fugene-6 reagent (Roche). Measurements were performed 36-48 hours following transfection.

Zn/Ringer Superfusion:

Cells were superfused with a Ringer's solution containing: 120 mM NaCl, 5 mM KCl, 20 mM glucose, 10 mM Hepes, 0.5 mM MgCl, 1.8 mM CaCl₂ at pH 7.4. For depolarization experiments aimed to fully activate the LTCC, 45 mM NaCl were replaced with 45 mM KCl. ZnSO₄ was added at the indicated concentration to this solution.

Fluorescence and Imaging Measurements:

Cells were incubated for 30 minutes with 5 μM of the ratiometric dye Fura-2-AM (TEF Labs) in 0.1% BSA-sodium Ringer's solution. Fura-2-AM binds zinc with very high affinity. After incubation, the cells were washed in Ringer's solution and the cover glass mounted on a holder that allowed the perfusion of liquids into the cells. Signals resulting from Ca⁺² or Zn⁺² changes were distinguished by using the intracellular zinc chelator TPEN. Changes in intracellular cation concentration were monitored by a single cell imaging system, consisting of a Zeiss Axiovert 100, inverted microscope, Polychrome II monochromator (TILL Photonics) and a cooled CCD (PCO). The data was acquired and processed by Axon Imaging Workbench 2, Excel and KleidaGraph. Fura-2 was excited at 340 nm and 380 nm and the image obtained with a 510 nm long-pass filter. The data was analyzed post acquisition, by selecting single cells (30-50) from the field of view and recording their activity.

Western Blot:

Western Blots were carried out in the classical method described in Molecular Cloning—A laboratory manual by Sambroock et al., Cold Spring Harbor Laboratory Press, 2^{nd} edition, 1989.

### Example 1

Fura-2 Fluorescence is Dependent on Zn²⁺ Concentration

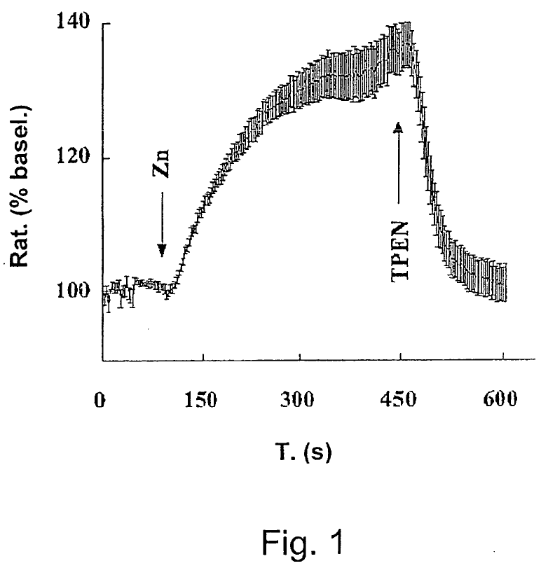

To measure the accumulation of intracellular zinc, HEK 293 cells were superfused with Ca-free, zinc-containing Ringer solution, and the release of Fura-2 was evaluated. Fura-2 is a highly sensitive probe for changes in intracellular Zn²⁺ concentration. Addition of TPEN (tetrakis-(2-pyridylmethyl)ethylenediamine), a membrane permeable heavy metal chelator, lowered the fluorescence signal (FIG. 1).

### Example 2

Zn²⁺ Influx is Inhibited by a L-Type Ca Channel Blocker

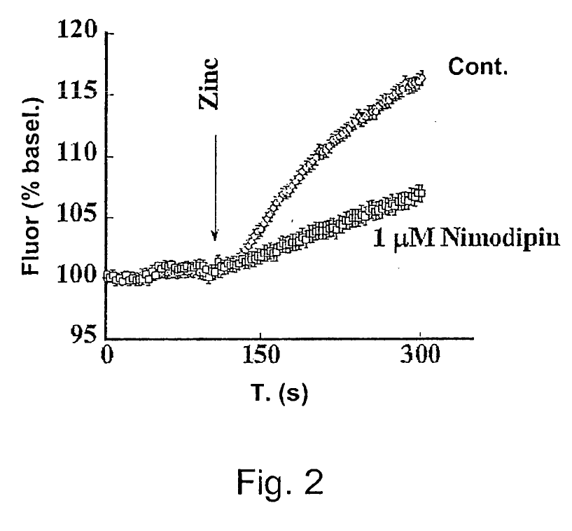

HEK 293 cells were exposed to 400 μM zinc with or without nimodipine, a_dihydropyridine that acts as a L-type Ca channel (LTCC) blocker. The LTCC blocker inhibited the influx of zinc into the cells (FIG. 2).

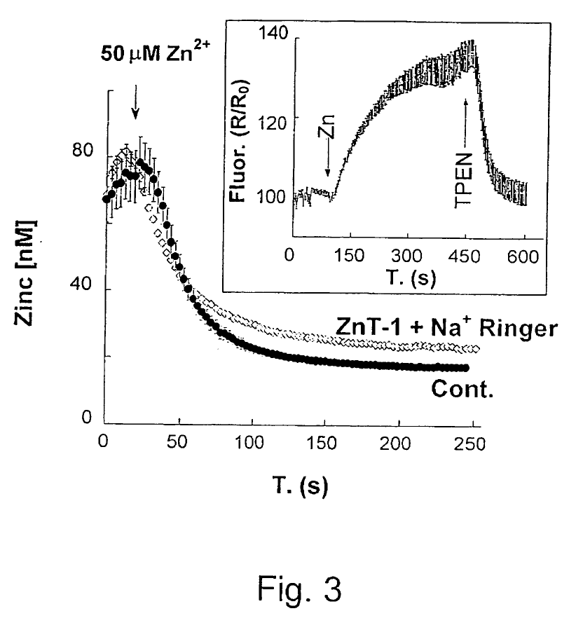

In contrast, the results shown in FIG. 3, where cells were loaded with Fura-2 and [Zn]ᵢ was monitored, demonstrate that Zinc efflux in cells transfected with ZnT-1 was similar to cells that were not transfected. This result suggests that ZnT-1 does not regulate Zinc efflux.

### Example 3

Expression of ZnT-1 Reduces Zinc Accumulation

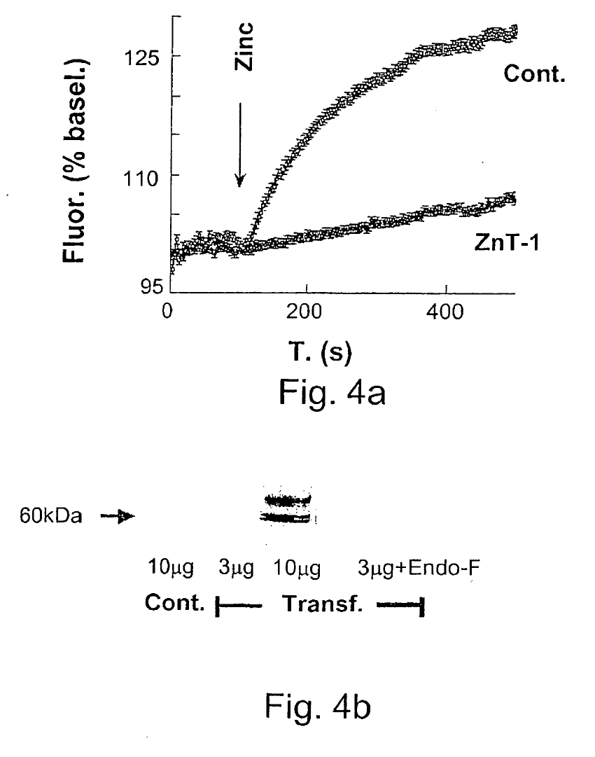

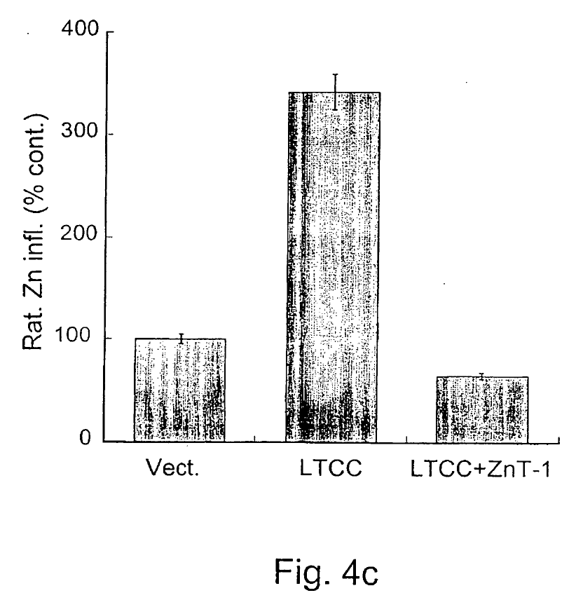

Control and ZnT-1 transfected HEK 293 cells were exposed to 400 μM zinc, and the rate of zinc influx was monitored over time (FIG. 4a-b). The rate of zinc influx was attenuated in the presence of ZnT-1 to levels comparable to those seen in FIG. 2, where the attenuation was caused by nimodipine. This result suggests that ZnT-1 functions in a manner similar to nimodipine, i.e., by blocking the LTCC.

Rates of zinc influx into HEK293 cells transfected with LTCC only or co-transfected with LTCC and ZnT-1 were measured. The histogram in FIG. 4c shows that there is a 5 fold reduction in rate of zinc permeation through LTCC in cells co-transfected with LTCC and ZnT-1 versus. LTCC alone

### Example 4

ZnT-1 Expression Inhibits Zn²⁺ Influx into Cells

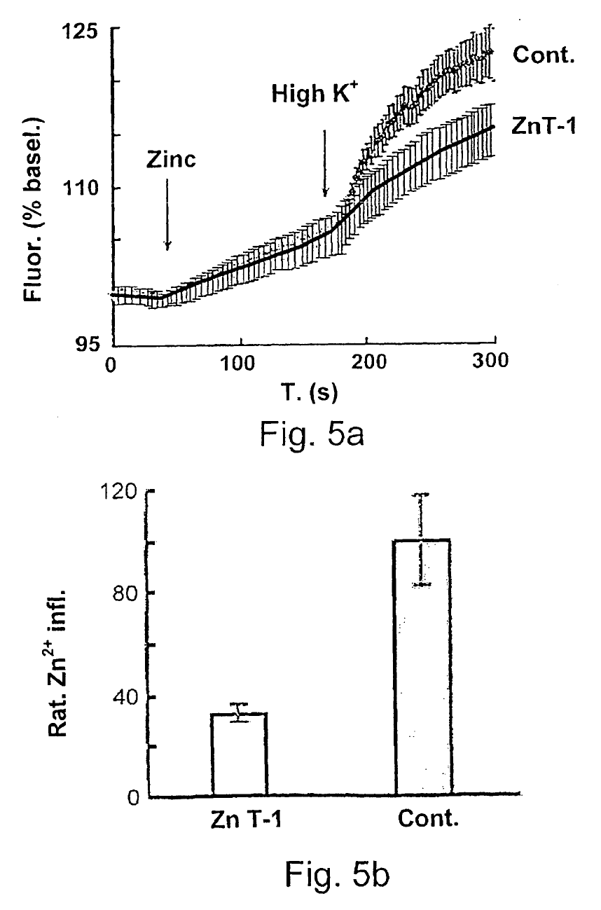

PC12 cells are a very good model for studying the activity of the LTCC. Control and ZnT-1 transfected PC12 cells, expressing endogenous L-type Ca channels, were superfused with Ringer solution containing 400 μM Zn²⁺ (FIG. 5a). The cells were then depolarized by high K⁺ in the presence of zinc. After depolarization, the rate of zinc influx was three-fold slower in cells expressing ZnT-1 vs. control cells (FIG. 5b).

### Example 5

Co-Expression of ZnT-1 and L-Type Ca Channels in HEK 293 Cells Reduces Zinc Influx.

Cells were transfected with L-type Ca channels (LTCC) alone or in combination with ZnT-1, and zinc influx was compared to control non-transfected cells. As depicted in FIG. 6, transfection with LTCC increased the zinc influx about three-fold. Co-expression of LTCC and ZnT-1 caused reduction in the zinc influx to levels comparable to control (FIG. 6). This result indicates that ZnT-1 acts as an inhibitor of divalent cation influx mediated by LTCC.

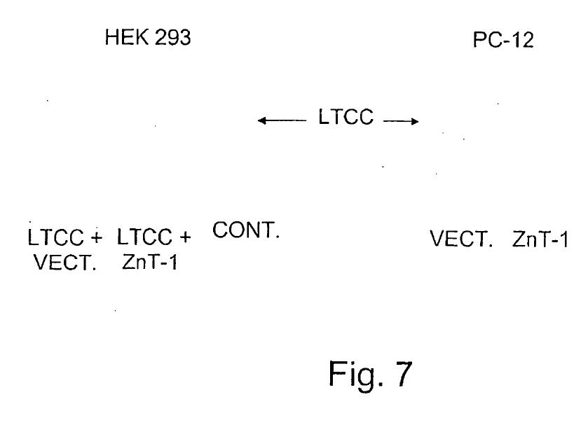

Interestingly, LTCC expression is not changed by ZnT-1 expression.HEK 293 cells transfected with LTCC, and PC12 cells endogenously expressing LTCC were transfected with ZnT-1 plasmid, and LTCC expression level was analyzed by immunoblots (FIG. 7). Neither cell type showed changes in LTCC expression in the presence or absence of ZnT-1. Thus, ZnT-1 putative regulation of LTCC is likely to be at the functional level, and not at the expression level.

### Example 6

ZnT-1 Expression Inhibits Ca²⁺ Influx into Cells

Control and ZnT-1 transfected PC12 cells, expressing endogenous L-type Ca channels, were superfused with Ca-free Ringer solution. After addition of 1.8 mM calcium, the cells were depolarized by high K⁺ for opening of the LTCC. The results shown in FIG. 8 demonstrate that ZnT-1 inhibits the rate of Ca influx.

### Example 7

ZnT-1 Expression is Induced by Zn in Cultured Brain Astrocytes

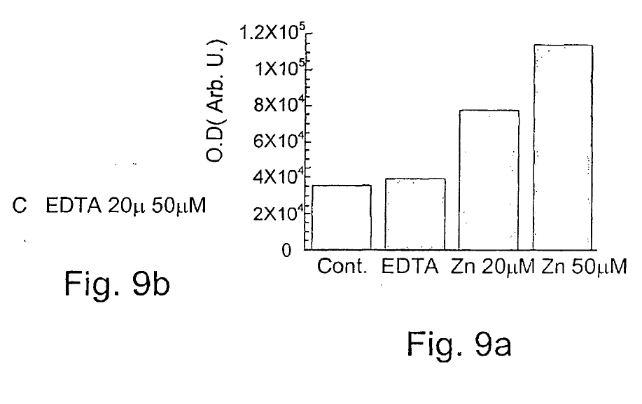

In astrocytes, zinc influx is mainly mediated by the LTCC. Astrocytes were treated with sub-lethal amounts (20 and 50 μM) of Zn for 12 hours, and ZnT-1 expression evaluated by Western Blot (FIG. 9b). The histogram shows the level of ZnT-1 expression in cells treated with different concentrations of Zn or with EDTA (FIG. 9a). ZnT-1 expression is induced by Zn⁺² in a dose-dependent manner.

### Example 8

Zinc Influx in Cells Induced to Express ZnT-1 Protein is Reduced in a Concentration-Dependent Manner

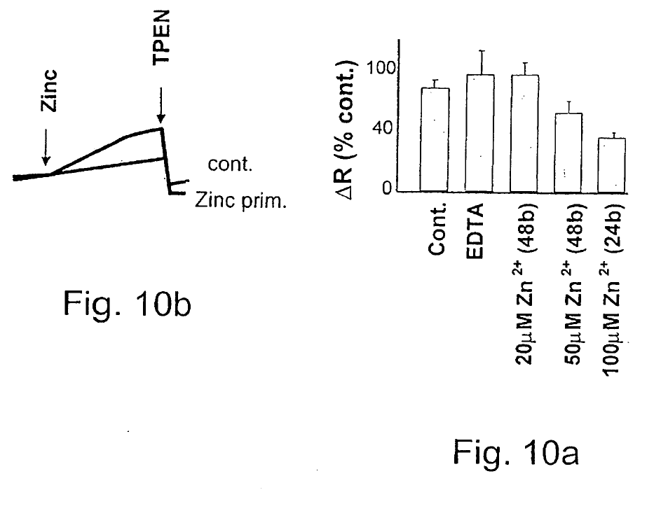

Astrocytes were pre-treated with 20, 50 or 100 μM of zinc for 24 or 48 hours, and subsequent rates of zinc influx were determined. These different treatments were comparable, since the induction of ZnT-1 was stable. Relative rates of zinc influx are represented by a histogram (FIG. 10a). Zinc treatment resulted in a reduction of zinc influx. The reduced zinc influx correlates with the increase in ZnT-1 expression, observed in FIG. 9. These data strongly suggests that the expression level of ZnT-1 can be controlled by non-harmful substances such as zinc, which consequently result in the reduced activity of the LTCC. In sum, ZnT-1 can control the activity of LTCC.

### Example 9

ZnT-1 and LTCC Physically Interact

Immunopercipitation using ZnT-1 antibody followed by Western blot with LTCC antibody was performed. As shown in FIG. 11, co-expression of the two proteins in HEK 293 cells resulted in much higher co-immunopercipitation. A similar effect is seen in mouse cardiac tissue, that expresses both ZnT-1 and the LTCC. Therefore, these results indicate that there is a physical interaction between ZnT-1 and LTCC, which is probably a cause or the result of the regulatory effect of ZnT-1 on LTCC.

1. ZnT-1 for use as a cellular L-type calcium channel (LTCC) blocker, which blocker has the properties of:
(a) a modulator of LTCC function; and
(b) a modulator of intracellular calcium concentration.

2. ZnT-1 of claim 1, for use as an LTCC blocker in the treatment or prevention of cardiovascular diseases and related indications.

3. ZnT-1 of claim 1, for use in the treatment or prevention of hypertension and stroke.

4. ZnT-1 of claim 1, wherein said ZnT-1 is active in any one of vascular, brain or heart cells.

5. A composition for blocking L-type calcium channels, comprising as active agent ZnT-1, and optionally comprising pharmaceutically acceptable adjuvant, carrier, diluent or excipient.

6. The composition of claim 5, for the treatment or prevention of cardiovascular diseases and related indications.

7. The composition of claim 6, for the treatment or prevention of hypertension and stroke.

8. A method of treating cardiovascular disorders in a patient in need of such treatment, comprising administering to said patient ZnT-1 or the composition comprising as active agent ZnT-1, and optionally comprising pharmaceutically acceptable adjuvant, carrier, diluent or excipient.

9. Use of ZnT-1 in the preparation of a pharmaceutical composition for the treatment or prevention of cardiovascular diseases and related indications, preferably said cardiovascular diseases are hypertension and stroke.

10. The method of claim 8, wherein said disorder comprises hypertention or stroke.
